(19) United States
(12) Patent Application Publication
(10) Pub. No.: US 2024/0350395 A1
(43) Pub. Date: Oct. 24, 2024

(54) HYALURONIC ACID (HA) FILLER COMPOSITION USING POLYETHYLENE GLYCOL (PEG) AND GLYCOLIC ACID (GA) AS CROSSLINKING AGENTS AND METHOD FOR PREPARING SAME

(71) Applicants: Dokyoung LEE, Cheongju-si, Chungcheongbuk-do (KR); Eunyoung LEE, Sejong (KR)

(72) Inventors: Dokyoung LEE, Cheongju-si, Chungcheongbuk-do (KR); Eunyoung LEE, Sejong (KR)

(21) Appl. No.: 18/685,577

(22) PCT Filed: Jul. 7, 2022
(86) PCT No.: PCT/KR2022/009837

§ 371 Date: Feb. 22, 2024

(30) Foreign Application Priority Data
Aug. 24, 2021 (KR) 10-2021-0111365

(51) Int. Cl. A61K 8/73; A61K 8/365; A61K 8/86; A61Q 19/08

(52) U.S. Cl. CPC A61K 8/735; A61K 8/365; A61K 8/86; A61Q 19/08

(57) ABSTRACT

The present invention relates to a hyaluronic acid (HA) filler composition using polyethylene glycol (PEG) and glycolic acid (GA) as crosslinking agents and a method for preparing same. The present invention is characterized by preparation through the crosslinking of hyaluronic acid (HA) with polyethylene glycol (PEG) and glycolic acid (GA). According to the present invention, polyethylene glycol (PEG) and glycolic acid (GA) are alternately added and stirred to crosslink liquefied hyaluronic acid particles, and viscoelasticity is controlled by adjusting the amounts of PEG and GA added, and thus, a filler composition that is elastically applicable to a treatment area is provided.

### TECHNICAL FIELD

The present invention relates to a filler composition for improving wrinkles, skin defects, and atrophic scars, more particularly to a filler composition in which polyethylene glycol (PEG) and glycolic acid (GA) are alternately bonded to liquefied hyaluronic acid (HA) for crosslinking and a method for preparing the same.

### BACKGROUND ART

Fillers are a substance similar to a skin tissue that are inserted into a specific area and used to improve wrinkles or correct contours by expanding a soft tissue. Also, it is called a dermal filler.

These fillers can be broadly classified into two types.

First, it is a filler that creates an enlargement effect by directly increasing the volume. The main ingredients of these skin fillers are made of substances such as collagen or hyaluronic acid.

Second, there are fillers, such as cross-linked textran, that have the effect of directly increasing volume and at the same time create a foreign body reaction over a certain period of time to induce autologous collagen formation for a long period of time or permanently, resulting in an enlargement effect.

The hyaluronic acid (HA), which is the main substance of the present invention, is a natural polymer that is abundant in the skin of animals, etc., and is a hydrophilic material that exists in the living body such as the epidermis, the cartilage, the vitreous humor of the eye, the synovial fluid of joints, etc.

7 to 8 g of the hyaluronic acid exists in the human body and plays an important role in maintaining the structure of the skin and its normal function.

However, the hyaluronic acid itself has limited viscosity and elasticity like water because the HA molecules are separated from each other, and is easily decomposed in vivo or under acid or alkali conditions, so that its use is limited (half-life in vivo is about 1 to 3 days).

Accordingly, efforts are being made to develop structurally stable hyaluronic acid derivatives. Recently, cross-linking agents such as BDDE, BDDA, and DVS are used to link HA molecules together, causing the molecules to clump together and gradually change them into a jelly-like form.

If the hyaluronic acid, that is not cross-linked, is putted into the body, it is quickly absorbed and has little viscosity and elasticity, so that it cannot even take shape.

In that respect, the cross-linking agent is an essential element in making hyaluronic acid filler.

There are three or four types of cross-linking agents currently used to make the hyaluronic acid fillers, including DVS (DiVinyl Sulfone), BCDI (Bis ethyl CarboDiimlde), and BDDE (ButaneDiol Diglycidyl Ether).

Among these, the BDDE is the most used these days, but it has the problem of leaving toxic residues, and the DVS (DiVinyl Sulfone), which was widely used as a crosslinking agent in the past, was withdrawn due to many side effects.

In other words, unlike the hyaluronic acid (HA), which is a natural ingredient, these cross-linking agents are chemical ingredients, so in some cases, they can cause side effects such as toxic reactions.

Also, in normal cases, it should exist in the form of ‘HA molecule-cross-linking agent-HA molecule’. However, there are cases where some cross-linking agents do not play a cross-linking role, and this amount is called cross-linking agent residual amount. There is a problem that the greater the residual amount, the greater the probability that the filler will cause inflammation or allergic reactions in the human body.

On the other hand, when the hyaluronic acid (HA) is bonded more tightly through cross-linking several times, it becomes more tightly packed. The one made soft after a few times is called monophasic hyaluronic acid fillers, and the one made hard after many times is called biphasic hyaluronic acid fillers.

Looking at products distributed on the market, representative monophasic hyaluronic acid fillers include Juvederm filler, Elravie filler, and e.p.t.q filler, and representative biphasic hyaluronic acid fillers include Restylane filler, Yvoire filler, and Perfecta filler.

It is impossible to decide which is better between the monophasic filler and the biphasic filler, and both have pros and cons.

In other words, the monophasic filler has very fine particles and cannot increase volume much, but it can create a natural effect because it maintains the shape thereof well and stays in the area well, so that it is mainly used in areas such as under the eyes.

On the other hand, the biphasic filler is good for adding volume because it has strong elasticity that allows it to bounce back even when subjected to external shock. In addition, the biphasic filler can also maintain shape thereof well, so that it is usually applied to areas that require a lot of volume, such as the nose, the front cheekbones, and the nasolabial folds.

Recently, a filler using PEG (polyethylene glycol), which is known to be purer than the BDDE, as a cross-linking agent has been released and are being marketed.

Recently, Matex Lab S.p.A. in Italy has been developed a filler using the PEG, which is said to be 30 times less toxic than the cross-linking agent BDDE, as a substitute.

For reference, the PEG is a substance approved by the USA FDA in 1990 and is known to have the characteristic of being completely decomposed.

However, as can be seen from “a filler composition for skin” (Korean Patent Laid-Open Publication No. 10-2019-0067653, Patent Literature 1), in case of the technology of the above-mentioned product, the weight ratio of hyaluronic acid (HA) is 6 to 18%, polyethylene glycol (PEG) is in a small amount of 0.5% to 4%, and water and hydroxide solution account for most of the remaining weight ratio. Accordingly, there is a limitation in obtaining the maximum benefits of the active ingredients of hyaluronic acid (HA) and polyethylene glycol (PEG).

Therefore, there is a demand for technology for filler compositions of various product lines that can be applied flexibly depending on the patient's skin condition and location, wherein while using the safe ingredient hyaluronic acid (HA) as much as possible, the duration of the effect can be extended at a lower cost, no cross-linking agent residue is generated, and viscoelasticity can be adjusted according to the composition ratio of each ingredient used as a cross-linking agent.

### PATENT LITERATURE

Patent Literature 1: Korean Patent Laid-Open Publication No. 10-2019-0067653 (Jun. 17, 2019)

### DISCLOSURE

### Technical Problem

The present invention is intended to solve the above problems of prior art, and is intended to provide a filler composition that can be applied flexibly depending on the area subjected to a procedure by alternately adding polyethylene glycol (PEG) and glycolic acid (GA) and performing stirring for crosslinking of liquefied hyaluronic acid particles and controlling viscoelasticity through adjustment of the amounts of PEG and GA added.

More specifically, the present invention is intended to provide a filler composition not only of which the viscosity and elasticity can be controlled through adjustment of the amounts of PEG and GA added but also which can last for a long period of time while maintaining the volume in the dermis.

In addition, the present invention is intended to facilitate a procedure by lowering the injection pressure.

The present invention is also intended to provide a filler composition that is slowly decomposed by hyaluronidase.

### Technical Solution

According to one aspect of the present invention so as to accomplish these objects, there is provided to a hyaluronic acid (HA) filler composition using polyethylene glycol (PEG) and glycolic acid (GA) as crosslinking agents, the filler composition being prepared by crosslinking hyaluronic acid (HA) using polyethylene glycol (PEG) and glycolic acid (GA).

In the above configuration, the hyaluronic acid (HA) is contained at 50% by weight, the polyethylene glycol (PEG) is contained at 15% to 40% by weight and the glycolic acid (GA) is contained at 10% to 35% by weight.

At this time, the hyaluronic acid (HA) is liquefied to have a viscosity of 1.60 to 2.0 m³/kg.

In addition, there is provided to a method for preparing a hyaluronic acid (HA) filler composition using polyethylene glycol (PEG) and glycolic acid (GA) as crosslinking agents, the method including preparing a crosslinked filler composition by alternately adding a remaining amount of polyethylene glycol (PEG) and glycolic acid (GA) to 50% by weight of a hyaluronic acid solution and performing stirring, wherein a weight ratio of polyethylene glycol (PEG) to glycolic acid (GA) is adjusted to 1:2.5 to 1.5 to increase viscosity or a weight ratio of polyethylene glycol (PEG) to glycolic acid (GA) is adjusted to 4 to 2:1 to increase elasticity.

In the above configuration, a crosslinked filler composition is prepared by alternately adding polyethylene glycol (PEG) and glycolic acid (GA) to the hyaluronic acid solution having a viscosity of 1.60 to 2.0 m³/kg and performing stirring for 12 to 18 hours while maintaining a temperature of 55° C. to 75° C.

### Advantageous Effects

According to the present invention, there is provided a filler composition that can be applied flexibly depending on the area subjected to a procedure by alternately adding polyethylene glycol (PEG) and glycolic acid (GA) and performing stirring for crosslinking of liquefied hyaluronic acid particles and controlling viscoelasticity through adjustment of the amounts of PEG and GA added.

More specifically, there is provided a filler composition not only of which the viscosity and elasticity can be controlled through adjustment of the amounts of PEG and GA added but also which can last for a long period of time while maintaining the volume in the dermis.

In addition, a procedure is facilitated since the injection pressure is low.

There is also provided a filler composition that is slowly decomposed by hyaluronidase.

### BRIEF DESCRIPTION OF DRAWINGS

The above and other objects, features and advantages of the present invention will be more apparent from the following detailed description taken in conjunction with the accompanying drawings, in which:

FIG. 1 is a graph illustrating the viscosity and elasticity of Examples and Comparative Examples in the present invention;

FIG. 2 is a graph illustrating the tangent delta of Examples and Comparative Examples in the present invention;

FIG. 3 is a graph illustrating the complex viscosity of Examples and Comparative Examples in the present invention; and

FIG. 4 is photographs of a patient subjected to a procedure using a composition of Example 1 in the present invention over time.

### BEST MODE

### Mode for Invention

In a case where the viscosity of a filler is high, the filler takes the form of a gel, is soft and tender, and thus is naturally positioned in the skin. However, as it relatively does not feel hard, it is not adequate to offer a definite sense of volume.

In a case where the elasticity of a filler is high, the filler takes the form of particles, feels a little hard, thus is adequate to offer a sense of volume and maintains the shape well after once used in a procedure.

The maintenance period of a filler is longer as the elasticity of the filler is higher, but there is a problem that the surface is uneven and layers are formed on the skin depending on the skill level in a case where the elasticity of a filler is excessively high.

In the present invention, polyethylene glycol (PEG) and glycolic acid (GA) are used as crosslinking agents to facilitate control of the viscosity and elasticity of a filler, and fillers can be prepared and used in a customized manner to the skin areas.

Hereinafter, a hyaluronic acid (HA) filler composition using polyethylene glycol (PEG) and glycolic acid (GA) as crosslinking agents and a method for preparing the same of the present invention will be described.

In the present invention, a filler composition is prepared by crosslinking hyaluronic acid (HA) using polyethylene glycol (PEG) and glycolic acid (GA).

As mentioned above, hyaluronic acid itself almost does not exhibit viscosity and elasticity like water since the HA molecules are separated from each other and use thereof is limited since it is easily decomposed in vivo or under acid or alkali conditions.

In Patent Literature 1, PEG is known as a crosslinking agent exhibiting low toxicity, and PEG cannot be contained in a large amount since the elasticity of a filler is improved but the filler becomes hard later as the amount of PEG used increases.

The inventors of this application have found out that when glycolic acid (GA), which is known to be harmless to the human body as a raw material of cosmetics, is added as a raw material for crosslinking of hyaluronic acid, the control of viscosity is facilitated so that the hyaluronic acid filler can be applied to various skin areas.

In addition, the inventors of this application have found out that when GA is added, the complex viscosity increases and the tangent delta value decreases, resulting in better shape maintenance.

At this time, as the most preferable composition, it is appropriate that hyaluronic acid (HA) is contained at 50% by weight, polyethylene glycol (PEG) is contained at 15% to 40% by weight, and glycolic acid (GA) is contained at 10% to 35% by weight in the entire composition.

In a case where polyethylene glycol (PEG) is contained at less than 15% by weight, the elasticity becomes too low, making it difficult to maintain the shape through filler injection.

On the other hand, in a case where polyethylene glycol (PEG) is contained at more than 40% by weight, the filler composition becomes hard and the injection procedure is not easy.

In a case where glycolic acid (GA) is contained at less than 10% by weight, the effect obtained by GA addition is reduced and the shape cannot be maintained for a long period of time. In a case where glycolic acid (GA) is contained at more than 35% by weight, the viscosity is relatively high and the procedure is not easy.

In a case where hyaluronic acid is contained at less than 50% by weight, volume formation becomes difficult. On the other hand, in a case where hyaluronic acid is contained at more than 50% by weight, the viscosity decreases to cause a flowing phenomenon, whereby the long-lasting properties are reduced.

The preparation of a filler composition for this includes preparing a crosslinked filler composition by alternately adding the remaining amount of polyethylene glycol (PEG) and glycolic acid (GA) to 50% by weight of a hyaluronic acid solution and performing stirring, in which the weight ratio of polyethylene glycol (PEG) to glycolic acid (GA) is adjusted to 1:2.5 to 1.5 to increase the viscosity and the weight ratio of polyethylene glycol (PEG) to glycolic acid (GA) is adjusted to 4 to 2:1 to increase the elasticity.

In a case where the content of glycolic acid is higher than the content of polyethylene glycol, the viscosity can be relatively increased and the filler composition is suitable for application to areas such as lips and cheeks.

On the other hand, in a case where the content of glycolic acid is lower than the content of polyethylene glycol, the filler composition is proper for procedures on areas such as the bridge of the nose and the tip of the chin where the volume is required to be definite, firm, and clearly formed or procedures to fill defective skin such as deeply engraved wrinkles.

In a case where the content of glycolic acid and the content of polyethylene glycol are similar, the viscosity and the elasticity are properly balanced and the filler composition is suitable for procedures on the cheekbones of the face or the forehead area just below the front hair.

At this time, it is preferable to prepare a crosslinked filler composition by alternately adding polyethylene glycol (PEG) and glycolic acid (GA) to the hyaluronic acid solution having a viscosity of 1.60 to 2.0 m³/kg and performing stirring for 12 to 18 hours while maintaining a temperature of 55° C. to 75° C.

In the preparation process, the dissolution time of raw materials increases and the productivity decreases in a case where the temperature is less than 55° C., and evaporation occurs and the control of viscosity is difficult in a case where the temperature exceeds 75° C.

The effect obtained by a procedure does not last for a long period of time in a case where the viscosity of the hyaluronic acid solution is less than 1.6, and the recipient feels irritation caused by a foreign matter and injection is difficult in a case where the viscosity of the hyaluronic acid solution exceeds 2.0.

Meanwhile, the proper molecular weight of PEG is between 180 and 700. In a case where the molecular weight exceeds 1000, PEG becomes solid at room temperature and requires an additional melting process, and in a case where the molecular weight is less than 180, there is a problem that the purity of PEG decreases.

### EXAMPLES

Hereinafter, Examples of the present invention will be described.

### Example 1

An aqueous sodium hydroxide solution (0.1%) was prepared by adding sodium hydroxide to ultrapure water at 60° C., sodium hyaluronate (NaHA, Mw=0.5 to 3 MDa) was added to the prepared aqueous sodium hydroxide solution in a weight to be ⅕ of the weight of the aqueous sodium hydroxide solution, and then stirring was performed at a medium speed of 400 rpm for 16 hours while the temperature was maintained, thereby preparing a hyaluronic acid solution having a viscosity of 1.8 m³/kg.

Polyethylene glycol (PEG) having an average molecular weight of 180 to 700 Mw and glycolic acid (GA) adjusted to have a pH of 6.5 were prepared and weighed so that the hyaluronic acid solution was 50% by weight, polyethylene glycol (PEG) was 15% by weight, and glycolic acid (GA) was 35% by weight. Polyethylene glycol (PEG) and glycolic acid (GA) were alternately added by ⅕ of the weight at 5-minute intervals and stirring was performed to prepare a crosslinked filler composition.

### Example 2

The same process as in Example 1 was carried out, but the weight was measured so that polyethylene glycol (PEG) was 20% by weight and glycolic acid (GA) was 30% by weight.

### Example 3

The same process as in Example 1 was carried out, but the weight was measured so that polyethylene glycol (PEG) was 25% by weight and glycolic acid (GA) was 25% by weight.

### Example 4

The same process as in Example 1 was carried out, but the weight was measured so that polyethylene glycol (PEG) was 30% by weight and glycolic acid (GA) was 20% by weight.

### Example 5

The same process as in Example 1 was carried out, but the weight was measured so that polyethylene glycol (PEG) was 35% by weight and glycolic acid (GA) was 15% by weight.

### Example 6

The same process as in Example 1 was carried out, but the weight was measured so that polyethylene glycol (PEG) was 40% by weight and glycolic acid (GA) was 10% by weight.

### Comparative Example 1

The same process as in Example 1 was carried out, but the weight was measured so that polyethylene glycol (PEG) was 42% by weight and glycolic acid (GA) was 8% by weight.

### Comparative Example 2

The same process as in Example 1 was carried out, but the weight was measured so that polyethylene glycol (PEG) was 45% by weight and glycolic acid (GA) was 5% by weight.

### Comparative Example 3

The same process as in Example 1 was carried out, but the weight was measured so that polyethylene glycol (PEG) was 13% by weight and glycolic acid (GA) was 37% by weight.

### Comparative Example 4

The same process as in Example 1 was carried out, but the weight was measured so that polyethylene glycol (PEG) was 10% by weight and glycolic acid (GA) was 40% by weight.

### Comparative Example 5

The same process as in Example 1 was carried out, but the weight was measured so that the hyaluronic acid solution was 48% by weight, polyethylene glycol (PEG) was 25% by weight, and glycolic acid (GA) was 27% by weight.

### Comparative Example 6

The same process as in Example 1 was carried out, but the weight was measured so that the hyaluronic acid solution was 45% by weight, polyethylene glycol (PEG) was 25% by weight, and glycolic acid (GA) was 30% by weight.

### Comparative Example 7

The same process as in Example 1 was carried out, but the weight was measured so that the hyaluronic acid solution was 52% by weight, polyethylene glycol (PEG) was 25% by weight, and glycolic acid (GA) was 23% by weight.

### Comparative Example 8

The same process as in Example 1 was carried out, but the weight was measured so that the hyaluronic acid solution was 55% by weight, polyethylene glycol (PEG) was 25% by weight, and glycolic acid (GA) was 20% by weight.

### Comparative Example 9

The same process as in Example 1 was carried out, but the weight was measured so that the hyaluronic acid solution was 55% by weight and polyethylene glycol (PEG) was 50% by weight.

### Comparative Example 10

The same process as in Example 1 was carried out, but the weight was measured so that the hyaluronic acid solution was 55% by weight and glycolic acid (GA) was 50% by weight.

### <Experimental Example 1> Experiment for Viscoelasticity Measurement

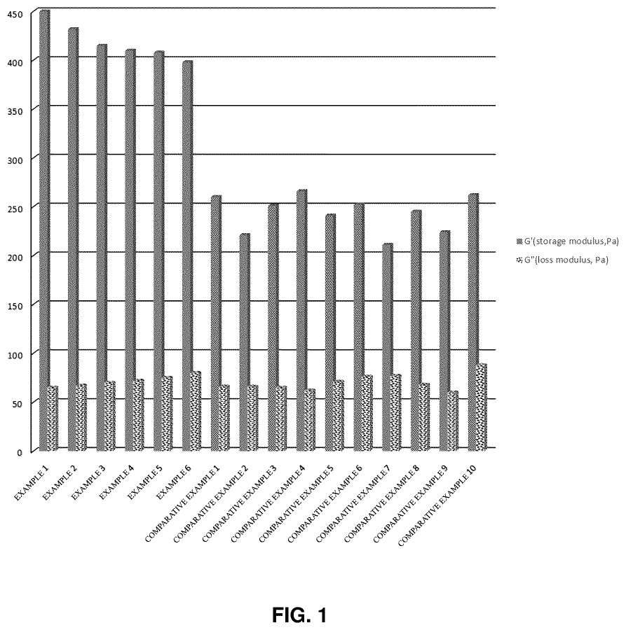

The elasticity (storage modulus), viscosity (loss modulus), tangent delta (tan δ) value, and complex viscosity of each sample were measured using a rotational rheometer (TA instrument Ltd., DHR-1), and the measurement results are illustrated in FIGS. 1 to 3.

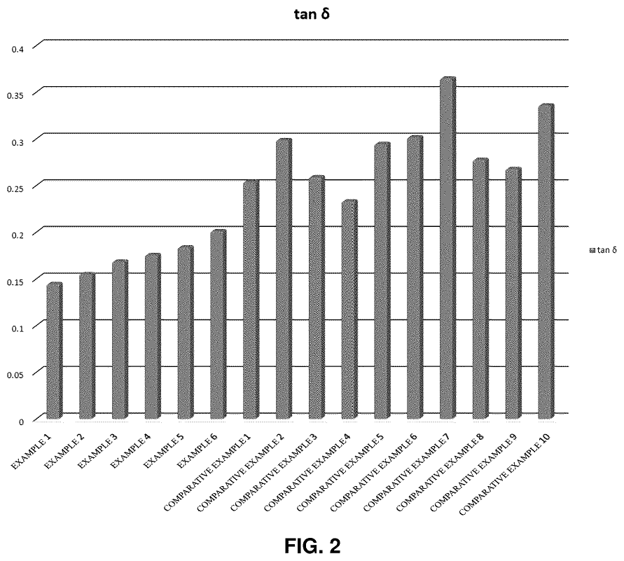

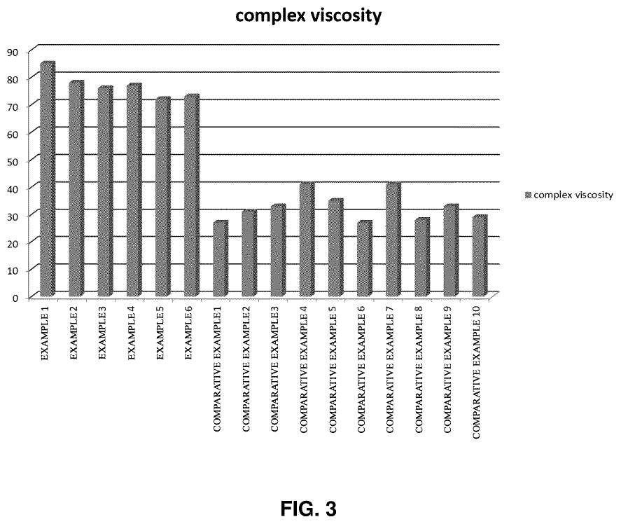

FIG. 1 illustrates elasticity and viscosity, FIG. 2 illustrates tangent delta, and FIG. 3 illustrates complex viscosity.

A numerical value that can be used to determine the degree of hardness is the tangent delta value, which is defined as the value obtained by dividing the viscosity coefficient by the modulus of elasticity. As this ratio, usually expressed as G″/G′, is close to 1 or greater than 1, the filler composition can be determined to exhibit high viscosity and favorable flowing properties. As this ratio is as small as less than 1, elasticity is dominant over viscosity and the filler composition is determined to be highly elastic and less likely to flow.

A large number of commercial fillers have a tangent delta value of less than 1, and commercial fillers that last for a long period of time while maintaining the volume in the dermis usually have a tangent delta value of 0.5 or less.

As a result of measuring the tangent delta value, it has been confirmed that Examples have harder properties than Comparative Examples and can last for a long period of time while maintaining the volume in the dermis.

As a result of measuring the complex viscosity, the complex viscosity of Examples is higher than that of Comparative Examples.

### <Experimental Example 2> Experiment for Injection Pressure Measurement

The prepared sample was filled into a syringe, a needle was fitted into the syringe, the sample was extruded at a speed of 5 mm/min, and then data was obtained using a tensile strength meter (JSV-1000, Jisco). From the data, the 5 mm point was set as point 1 after a load was applied to the sample and the point 5 mm before the point where the sample entirely flowed out and the load rose rapidly was set as point 2, and the average value of the two points was calculated and presented in the table below. (Unit: N)

TABLE 1

| Item | Injection pressure |
|---|---|
| Example 1 | 21.2 |
| Example 2 | 20.9 |
| Example 3 | 20.5 |
| Example 4 | 19.9 |
| Example 5 | 19.5 |
| Example 6 | 19.3 |
| Comparative Example 1 | 33.2 |
| Comparative Example 2 | 30.2 |
| Comparative Example 3 | 26.8 |
| Comparative Example 4 | 27.2 |
| Comparative Example 5 | 26.3 |
| Comparative Example 6 | 25.2 |
| Comparative Example 7 | 24.2 |
| Comparative Example 8 | 24.5 |
| Comparative Example 9 | 25.3 |
| Comparative Example 10 | 45.2 |

As a result of the experiment, it has been confirmed that the injection pressure of Examples is significantly lower than that of Comparative Examples.

### <Experimental Example 3> Experiment for Decomposition Ability Measurement

In a case where crosslinked hyaluronic acid (HA) is injected into the body, the crosslinked hyaluronic acid undergoes a direct decomposition attack by hyaluronidase and is decomposed.

By directly adding hyaluronidase to a sample, the decomposition tendency can be examined and resistance to the enzyme can be measured.

A decomposition ability test was conducted using the samples of Examples and Comparative Examples as test pieces and the materials and methods below.

Among the test solutions, a buffer was prepared by weighing 4.05 g of sodium chloride, 0.72 g of monohydrogen phosphate, and 0.31 g of dihydrogen phosphate and dissolving the reagents in distilled water, and an HADase stock solution was prepared by placing 125 mg of hyaluronidase (Sigma Aldrich, Bovine origin, H1136) in a 10 mL volumetric flask and massing up the volumetric flask with the buffer.

Then, 1 mL of the HADase stock solution was taken at HADase 2000 Units/mL, diluted with 4 mL of the buffer, and filtered before use.

For the experiment, about 0.5 g of sample was taken and placed in a glass syringe, centrifugation was performed for 5 minutes at 3000 rpm, the liquid was collected downward, bubbles were removed, centrifugation was then performed, a rubber stopper was then inserted, and the front part of the syringe was separated.

After 200 μl of HADase 200 Units/mL was gradually added together with air, the front part was recombined, and samples were taken by three after 12 and 24 hours while the reaction was conducted in an incubator at 37° C. The decomposition rate was calculated using the equation below, and the results (average value for three times) are presented in Table 2 below.

$$Decomposition rate = ( A - B ) / C * 100$$

A: Amount of decomposed liquid (including enzyme and decomposed HA)
B: Amount of enzyme added
C: Amount of crosslinked HA initially added

TABLE 2

| | Decomposition rate (%) | |
|---|---|---|
| Item | After 12 hours | After 24 hours |
| Example 1 | 6.6 | 39.2 |
| Example 2 | 7.2 | 39.5 |
| Example 3 | 7.6 | 40.1 |
| Example 4 | 6.9 | 40.3 |
| Example 5 | 7.3 | 42.5 |
| Example 6 | 7.5 | 43.3 |
| Comparative | 12.5 | 54.5 |
| Example 1 | | |
| Comparative | 15.7 | 62.5 |
| Example 2 | | |
| Comparative | 32.5 | 72.5 |
| Example 3 | | |
| Comparative | 33.4 | 70.5 |
| Example 4 | | |
| Comparative | 18.5 | 52.5 |
| Example 5 | | |
| Comparative | 19.6 | 58.6 |
| Example 6 | | |
| Comparative | 21.3 | 62.5 |
| Example 7 | | |
| Comparative | 23.3 | 58.2 |
| Example 8 | | |
| Comparative | 45.5 | 89.9 |
| Example 9 | | |
| Comparative | 38.8 | 82.5 |
| Example 10 | | |

As a result of the experiment, it can be seen that the decomposition in Examples progresses more slowly than the decomposition in Comparative Examples.

### <Experimental Example 4> Experiment for Wrinkle Improvement Measurement

After the samples of Examples and Comparative Examples were injected into the superficial dermis, the severity of wrinkles (WSRS average value, wrinkle severity rating scale) was measured for six months, and the results are presented in the table below.

TABLE 3

| | Before | After two | After four | After six |
|---|---|---|---|---|
| Item | procedure | months | months | months |
| Example 1 | 3.33 | 2.54 | 2.60 | 2.78 |
| Example 2 | 3.33 | 2.55 | 2.75 | 2.80 |
| Example 3 | 3.33 | 2.54 | 2.61 | 2.82 |
| Example 4 | 3.33 | 2.53 | 2.63 | 2.88 |
| Example 5 | 3.33 | 2.55 | 2.67 | 2.82 |
| Example 6 | 3.33 | 2.55 | 2.68 | 2.79 |
| Comparative | 3.33 | 2.65 | 3.11 | 3.20 |
| Example 1 | | | | |
| Comparative | 3.33 | 2.72 | 3.12 | 3.19 |
| Example 2 | | | | |
| Comparative | 3.33 | 2.66 | 3.02 | 3.23 |
| Example 3 | | | | |
| Comparative | 3.33 | 2.65 | 2.99 | 3.20 |
| Example 4 | | | | |
| Comparative | 3.33 | 2.64 | 2.97 | 3.19 |
| Example 5 | | | | |
| Comparative | 3.33 | 2.68 | 2.89 | 3.08 |
| Example 6 | | | | |
| Comparative | 3.33 | 2.66 | 2.79 | 3.11 |
| Example 7 | | | | |
| Comparative | 3.33 | 2.61 | 2.88 | 3.08 |
| Example 8 | | | | |
| Comparative | 3.33 | 2.58 | 2.82 | 3.22 |
| Example 9 | | | | |
| Comparative | 3.33 | 2.54 | 2.77 | 3.21 |
| Example 10 | | | | |

As presented in the table above, it can be seen that in Examples, the effect is maintained well for six months after the procedure.

On the other hand, in Comparative Examples, it can be seen that the effect is not maintained well.

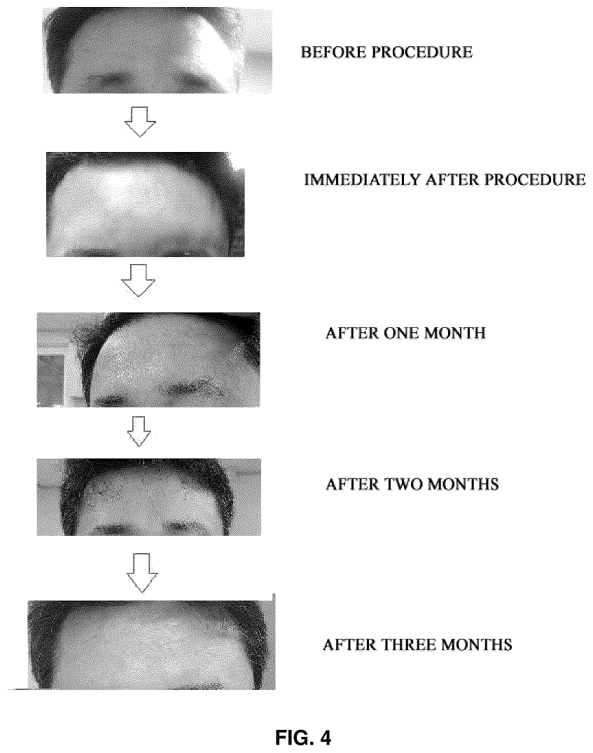

FIG. 4 illustrates the changes of one among the participants in Example 1 over time, and it can be seen that wrinkles are maintained in an improved state when time has passed after the procedure as well.

1. A hyaluronic acid (HA) filler composition using polyethylene glycol (PEG) and glycolic acid (GA) as crosslinking agents, the filler composition being prepared by crosslinking hyaluronic acid (HA) using polyethylene glycol (PEG) and glycolic acid (GA),
wherein the hyaluronic acid (HA) is contained at 50% by weight,
the polyethylene glycol (PEG) is contained at 15% to 40% by weight, and
the glycolic acid (GA) is contained at 10% to 35% by weight.

2. The hyaluronic acid (HA) filler composition using polyethylene glycol (PEG) and glycolic acid (GA) as crosslinking agents according to claim 1,
wherein the hyaluronic acid (HA) is liquefied to have a viscosity of 1.60 to 2.0 m³/kg.

3. A method for preparing a hyaluronic acid (HA) filler composition using polyethylene glycol (PEG) and glycolic acid (GA) as crosslinking agents, the method comprising:
preparing a crosslinked filler composition by alternately adding a remaining amount of polyethylene glycol (PEG) and glycolic acid (GA) to 50% by weight of a hyaluronic acid solution and performing stirring,
wherein a weight ratio of polyethylene glycol (PEG) to glycolic acid (GA) is adjusted to 1:2.5 to 1.5 to increase viscosity or
a weight ratio of polyethylene glycol (PEG) to glycolic acid (GA) is adjusted to 4 to 2:1 to increase elasticity.

4. The method for preparing a hyaluronic acid (HA) filler composition using polyethylene glycol (PEG) and glycolic acid (GA) as crosslinking agents according to claim 3,
wherein a crosslinked filler composition is prepared by alternately adding polyethylene glycol (PEG) and glycolic acid (GA) to the hyaluronic acid solution having a viscosity of 1.60 to 2.0 m³/kg and performing stirring for 12 to 18 hours while maintaining a temperature of 55° C. to 75° C.

1. A hyaluronic acid (HA) filler composition using polyethylene glycol (PEG) and glycolic acid (GA) as crosslinking agents, the filler composition being prepared by crosslinking hyaluronic acid (HA) using polyethylene glycol (PEG) and glycolic acid (GA),
wherein the hyaluronic acid (HA) is contained at 50% by weight,
the polyethylene glycol (PEG) is contained at 15% to 40% by weight, and
the glycolic acid (GA) is contained at 10% to 35% by weight.

2. The hyaluronic acid (HA) filler composition using polyethylene glycol (PEG) and glycolic acid (GA) as crosslinking agents according to claim 1,
wherein the hyaluronic acid (HA) is liquefied to have a viscosity of 1.60 to 2.0 m³/kg.

3. A method for preparing a hyaluronic acid (HA) filler composition using polyethylene glycol (PEG) and glycolic acid (GA) as crosslinking agents, the method comprising:
preparing a crosslinked filler composition by alternately adding a remaining amount of polyethylene glycol (PEG) and glycolic acid (GA) to 50% by weight of a hyaluronic acid solution and performing stirring,
wherein a weight ratio of polyethylene glycol (PEG) to glycolic acid (GA) is adjusted to 1:2.5 to 1.5 to increase viscosity or
a weight ratio of polyethylene glycol (PEG) to glycolic acid (GA) is adjusted to 4 to 2:1 to increase elasticity.

4. The method for preparing a hyaluronic acid (HA) filler composition using polyethylene glycol (PEG) and glycolic acid (GA) as crosslinking agents according to claim 3,
wherein a crosslinked filler composition is prepared by alternately adding polyethylene glycol (PEG) and glycolic acid (GA) to the hyaluronic acid solution having a viscosity of 1.60 to 2.0 m³/kg and performing stirring for 12 to 18 hours while maintaining a temperature of 55° C. to 75° C.
